(19) United States
(12) Patent Application Publication
(10) Pub. No.: US 2003/0207335 A1
(43) Pub. Date: Nov. 6, 2003

(54) Process for preparing monolayers and microarrays of biomolecules by using dendrimers

(76) Inventors: Hak-Sung Kim, Taejon (KR); Hyun-Chul Yoon, Seoul (KR); Mi-Young Hong, Taejon (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/460,808

(22) Filed: Jun. 11, 2003

Related U.S. Application Data
(62) Division of application No. 09/795,604, filed on Feb. 28, 2001, now abandoned.

(30) Foreign Application Priority Data
Jul. 15, 2000 (KR) 10-2000-0040829

(51) Int. Cl. G01N 33/53; B05D 3/00

(52) U.S. Cl. 435/7.1; 435/7.5; 427/2.11

(57) ABSTRACT

The present invention relates to a process for preparing monolayers and microarrays of biomolecules by reacting functionalized dendrimers on a solid surface with biomolecules such as proteins, antigens, antibodies, enzymes, ligands, receptors, and the like. The present invention can be widely applied to the areas including preparation of kits and biosensors for disease diagnosis and compound analyses using the ascribed biomolecules as target substances, and more recently, integrated high-throughput analyzing system such as development of protein chips.

### REFERENCE TO RELATED APPLICATIONS

The present invention claims priority as a divisional of U.S. application Ser. No. 09/795,604, filed Feb. 28, 2001, which in turn claims priority under 35 U.S.C. §119 to Republic of Korea Application Number 10-2000-0040829, filed Jul. 15, 2000.

### FIELD OF THE INVENTION

The present invention relates to a process for preparing monolayers and microarrays of biomolecules by using dendritic macromolecules, more specifically, to a process for preparing monolayers and microarrays of biomolecules by reacting functionalized dendrimers on a solid surface with biomolecules such as proteins, antigens, antibodies, enzymes, ligands, receptors, and the like.

### BACKGROUND OF THE INVENTION

Techniques for immobilizing biomolecules such as proteins, enzymes, antigens, antibodies, ligands, receptors and the like, or other macromolecular materials on the solid surface have been widely applied to; the areas including preparation of kits and biosensors for disease diagnosis and compound analyses by employing the ascribed biomolecules as target substances, and more recently, integrated high-throughput analyzing system such as the development of protein chips.

For the purposes described above, the techniques used routinely for immobilizing biomolecules were physical adsorption, electrochemical conjugation using electropolymerizable macromolecules, and covalent bonding. However, the method for physical adsorption of biomolecules onto the solid surface has revealed disadvantages that the quantity of immobilized biomolecules is restricted and adsorbed biomolecules are gradually released and/or inactivated. The method of electrochemical conjugation has also revealed disadvantages that the quantity of immobilized biomolecules cannot be strictly controlled as the biomolecules are impregnated in the network of electrically conductive macromolecules in the form of amorphous multilayer and consequently efficient interaction with other molecules cannot be achieved. The conventional immobilization techniques have limitations that the orientation of proteins needed for the biospecific interaction cannot be controlled and the stability of immobilized biomolecules cannot be maintained.

For the preparation of the said protein chips with sufficient sensitivity and specificity, the technique for preparing high-density microarrays of biomolecules on the solid surface is the prerequisite. The microarraying technique of biomolecules is in the developmental phase, and now, we are confronting the requirement of simple, efficient and more integrated techniques for microarray preparation. In this respect, microcontact printing technique which is simple in conducting, thus, does not need an expensive apparatus to prepare a microarray of biomolecules and gradually becomes popular. The technique has been developed by Dr. Whitesides at Harvard University, and was adopted with modifications by the present inventors.

### SUMMARY OF THE INVENTION

The present inventors have made an effort to immobilize biomolecules homogeneously and stably on the solid surface, thus, it has been discovered that homogeneous stable monolayers and microarrays of biomolecules in a high density can be constructed by conjugating biomolecules on a solid surface by employing dendrimers instead of thiol compound, an ink material, used in microcontact printing of prior art.

A primary object of present invention is, therefore, to provide a process for preparing biomolecular monolayers.

The other object of the invention is to process for preparing biomolecular microarrays.

### BRIEF DESCRIPTION OF THE DRAWINGS

The above object and features of the present invention will become apparent from the following descriptions given in conjunction with the accompanying drawing, in which:

FIG. 1 is a diagram of dendrimer structure containing amine chain-end groups.

FIG. 2 is a schematic representation of a process for preparing glycoprotein monolayers on dendrimer monolayers.

FIG. 3 is a schematic representation of a process for preparing avidin monolayer using dendrimers.

FIG. 4 is a schematic representation of a process for preparing a micropattern of dendrimers using microcontact printing method, and the resulting fluorescence image of the microarrayed avidin labeled with fluorescent materials.

### DETAILED DESCRIPTION OF THE PREFERRED EMBODIMENT

A process for preparing biomolecular monolayers using dendrimers comprises the steps of: reacting a metal surface or a glass surface with a solution of amine-terminated or succinimide-terminated alkanethiol for 1 to 2 hours to obtain a self-assembled monolayer; reacting the self-assembled monolayer with amine-terminated dendrimers or N-hydroxysuccinimide-modified, carboxyl-terminated dendrimers to give a dendrimer monolayer; and, reacting the dendrimer monolayer with a biomolecule of protein, antigen, antibody, enzyme, receptor or ligand.

Also, a process for preparing a microarray of biomolecules comprises the steps of: reacting a metal surface or a glass surface with a solution of alkanethiol or derivatized silane with amine reactive functionality to obtain a self-assembled monolayer; reacting the self-assembled monolayer with amine-terminated dendrimers to give micropattern of dendrimers; and, reacting the patterned dendrimers with a biomolecule of protein, antigen, antibody, enzyme, receptor or ligand.

The process for preparing monolayers and microarrays of biomolecules using dendrimers of present invention is illustrated in more detail by the following steps.

Step 1: Preparation of Self-Assembled Monolayer as a Platform for Preparing a Dendrimer Monolayer

A self-assembled monolayer is prepared by reacting a metal surface or a glass surface with a solution of alkanethiol or derivatized silane modified with succinimide for 1 to 2 hours. The metal includes gold, silver, copper and platinum, though silicon wafer with evaporated gold is the most preferred.

To prepare a monolayer of dendrimer containing amine groups, a solution of dithiopropionic acid bis-N-hydroxysuccinimide ester (alkanethiol having terminal succinimide) in dimethylsulfoxide (DMSO) is employed for the self-assembled monolayer (“SAM”) formation, while aqueous solution of cystamine dihydrochloride (alkanethiol having terminal amine groups) is employed to prepare dendrimer monolayers containing carboxyl groups modified with N-hydroxysuccinimide.

Step 2: Preparation of a Dendrimer Monolayer

A monolayer of dendrimer is prepared by reacting the self-assembled monolayer (alkanethiol or silane) with a methanolic solution of dendrimer having amine groups or a solution of dendrimer having carboxyl groups modified with N-hydroxysuccinimide for 30 minutes to 1 hour.

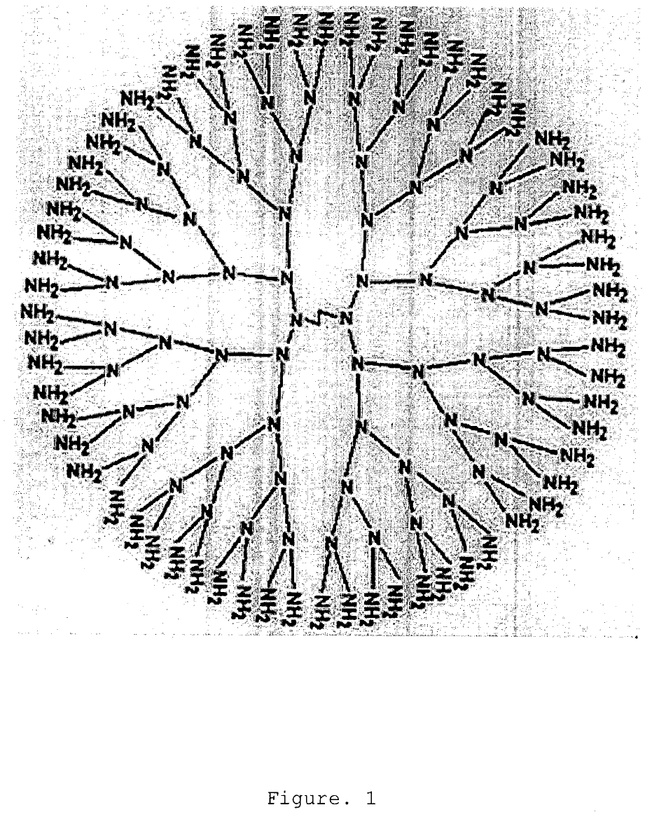

The amine-containing dendrimer includes G1, G2, G3, G4 and G5 dendrimers, though the spherical macromolecular G4 dendrimer containing 64 reactive amine groups may be preferably used. The structure of dendrimer containing amine groups is disclosed in FIG. 1. The said monolayer constructed with G4 dendrimers has high reactivity to aldehyde groups due to the reactive amine groups on its surface.

Dendrimers containing carboxyl groups modified with N-hydroxysuccinimide may be obtained by modifying carboxyl groups of dendrimer selected from the group consisting of G1.5, G2.5, G3.5, G4.5 and G5.5, preferably G3.5, with N-hydroxysuccinimide. Since the dendrimers containing carboxyl groups modified with N-hydroxysuccinimide are highly reactive to amine groups, they have high reactivity to the said self-assembled monolayer prepared; in Step 1. The dendrimers dissolved in alcohols such as ethanol or methanol, preferably methanol, in a concentration range of 0.01 to 0.1 mM, preferably 0.022 to 0.04 mM, are used, while the dendrimers containing carboxyl groups modified with N-hydroxysuccinimide are dissolved in a buffer solution before use.

Separately, monolayer of amine-terminated dendrimers may be directly constructed by immersing aldehyde silane-coated slide glass in a methanolic solution of amine-terminated dendrimers. After reaction for 2 hours, the monolayer may be reduced by sodium borohydride to enhance the stability of dendrimer monolayers.

Step 3: Preparation of Biomolecular Monolayers

Biomolecular monolayers are finally prepared by coupling biomolecules such as proteins, glycoproteins, antigens, antibodies, enzymes, receptors, and ligands to the dendrimer monolayer prepared above. The biomolecules containing sugar chains at the surface like immunoglobulins and other filycoproteins are reacted with dendrimer monolayers containing amine groups after sugar chains are oxidized with periodate to have aldehyde end groups, while other proteins containing amine groups like surface lysine residues are used as supplied to react with dendrimers modified with N-hydroxysuccinimide.

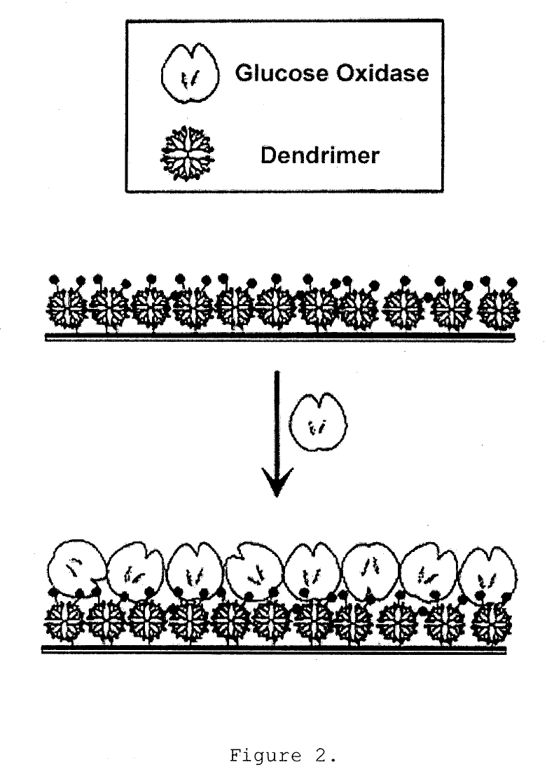

When the monolayer of a typical glycoprotein such as glucose oxidase and antibody is prepared by employing amine-terminated dendrimers, subsequent reduction reaction may be conducted by using borohydride compound for stabilization of imine linkage, and free aldehyde groups remained on their periphery of immobilized proteins may be blocked by ethanolamine treatment to avoid self-polymerization. As a typical example, the concentration of glucose oxidase immobilized on the protein monolayer was found to be 1.2×10⁻¹² to 1.7×10-12 mol/cm² of substrate, preferably, the maximum concentration of about 1.7×10-12 mol/cm². The schematic representation of a process for preparing glycoprotein monolayer using dendrimers of the invention is shown in FIG. 2.

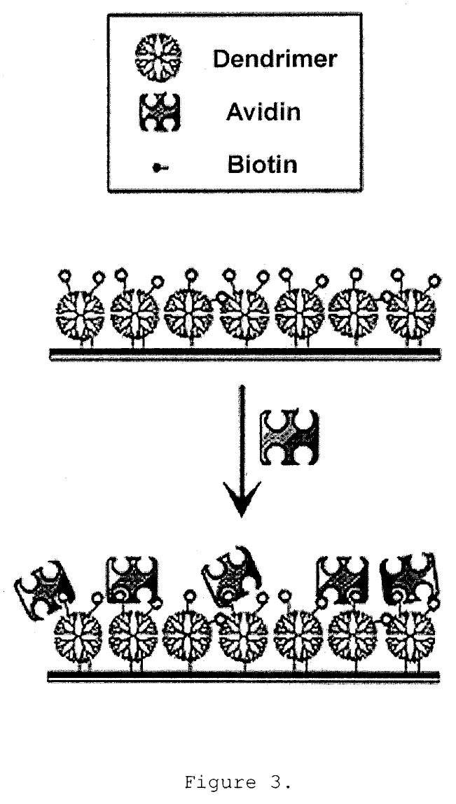

Furthermore, the present invention for preparing biomolecular monolayers interaction between biotin and avidin. First of all, monolayer is prepared using biotin-modified dendrimer molecules on the alkanethiol of derivatized silane self-assembled monolayer, and then avidin monolayer is prepared by reacting avidin to biotin-functionalities on dendrimer monolayer. Since free (unoccupied) biotin-binding sites on avidin monolayer are spatially oriented in the opposite direction after avidin binding reaction, biomolecular adlayer can be prepared by reacting biotin-modified biomolecules to preformed avidin monolayer. The schematic representation of a process for preparing avidin monolayer using dendrimers of the invention is shown in FIG. 3. The characteristics of monolayer formed above may be analyzed by ellipsometry, electrochemical method or fluorescence microscopy. The formation of avidin monolayer is verified by an electrochemical method. That is, biotinylated monolayer of dendrimers which is fully associated and covered with avidin molecules exhibited complete blockage characteristic from the bioelectrocatalytic testing with glucose oxidase.

The ellipsometric analysis of biomolecular monolayer based on the strong interaction between avidin and biotin manifested that the thickness of biomolecular monolayer on solid surface was comparable to the dimension of biomolecules, and electrochemical measurements of protein activity have demonstrated that the high-density protein monolayer has been formed. Also, fluorescence microscopic analysis of monolayers of biotinylated biomolecules demonstrated the formation of monolayer of biomolecules conjugated with fluorescent material on the solid surface.

The ascribed process for preparing biomolecular monolayers using dendrimers, compared to the conventional techniques, has advantages that homogeneous high-density monolayer of biomolecules can be prepared and the consideration of covalent bonding or orientation of proteins is not necessary. Thus, biomolecular monolayer of the invention can be widely applied to the development of diagnosis kits, biosensors, and protein chips, etc.

Step 4: Preparation of Biomolecular Microarrays

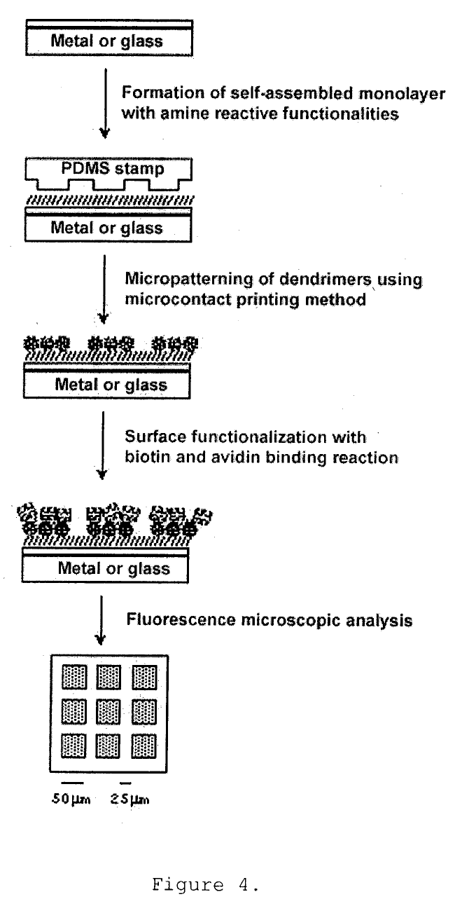

For the preparation of a biomolecular monolayer in a microarray format, the dendrimer monolayer described in Step 2 is micropatterned by using microcontact printing techniques as schematically shown in FIG. 4. The microcontact printing method is simple in conducting, thus, does not need an expensive apparatus to prepare a microarray of biomolecules and gradually becomes popular. The technique has been developed by Dr. Whitesides at Harvard University and was adopted with modifications by the present inventors. In the present invention, thiol compound, an ink material, used in microcontact printing is replaced with dendrimers to prepare a microarray of biomolecules in a more efficient way. The microarray of biomolecules on the micropatterned dendrimers can be prepared by using the same principles and procedures as described in Step 3. The fluorescence microscopic analysis of microarrayed avidin shown in FIG. 4 demonstrates that the present invention provides a process for preparing the microarray of biomolecules in the spatially ordered and site-specific manner with high resolution.

The present invention is further illustrated in the following examples, which should not be taken to limit the scope of the invention.

### EXAMPLE 1

### Preparation of Monolayer Using Poly(Amidoamine) Dendrimers

First, silicon wafer with evaporated gold was cleaned with ethanol dipping and a self-assembled monolayer was obtained by immersing the washed base substrate in a solution of 5 mM dithiopropionic acid bis-N-hydroxysuccinimide ester in DMSO for 2 hours. After washing with methanol, the self-assembled monolayer thus obtained was immersed in a solution of 0.022 mM amine-terminated dendrimer (Dendritech Inc., Midland) in methanol for 1 hour to obtain a dendrimer monolayer.

### EXAMPLE 2

### Preparation of Glucose Oxidase Monolayer

To prepare a glucose oxidase monolayer, periodate-treated glucose oxidase solution was reacted with the dendrimer monolayer prepared above for 30 minutes to 1 hour. To stabilize imine linkage formed in this reaction, reduction was conducted using sodium borohydride compound for 30 minutes, and free aldehyde groups remained on their periphery of immobilized enzymes were blocked by treatment with 10 mM ethanolamine for 30 minutes. The characterization of glucose oxidase monolayer on the film prepared above was performed by electrochemical method as follows: that is, the film with immobilized glucose oxidase was dipped into a buffer solution containing enzyme substrate and electron-transferring mediators, and then the concentration of resulting immobilized enzyme was measured by registering resulting bioelectrocatalyzed current by applying voltage of 250 mV. The concentration of immobilized glucose oxidase was estimated by kinetic simulation as 1.7×10⁻¹² mol/cm² and as high as ca. 80% of immobilized enzyme activity was retained after 20 day storage in a buffer solution under room temperature.

### EXAMPLE 3

### Preparation of Antigen/Antibody Monolayer on the Preformed Underlying Dendrimer Monolayer

The dendrimer monolayer was prepared analogously as in Example 1, and washed with distilled water. Then, the resulting dendrimer monolayer on the film was subject to following reactions to couple amine groups on the monolayer with biotin, that is, dendrimer monolayer was immersed in a solution of phosphate buffer containing 2 mg/ml biotin-N-hydroxysulfosuccinimide ester for 1 hour. After reaction, the film was washed with a buffer solution, and then avidin monolayer was prepared on the biotin-modified dendrimer monolayer by incubating with avidin molecules as follows: that is, biotin-modified dendrimer monolayer was immersed in a buffer solution containing glucose, glucose oxidase and ferrocene followed by applying a certain voltage on it to measure no change in bioelectrocatalytic current due to the complete blocking of electron transfer onto the electron conductive metal surface by avidin monolayer formed.

As a typical antigen, Ferritin was chosen, and its monolayer was prepared by incubating ascribed avidin monolayer in a solution of biotin-linked antigen (0.01 mg/ml) for 20 minutes, and then the resulting stable antigen monolayer was incubated with anti-ferritin antibody to the above antigen molecule to prepare antibody monolayer. Fluorescence microscopic observation of antibody monolayer made of FITC-labeled antibody demonstrated that antibody monolayer has been formed.

### EXAMPLE 4

### Preparation of Biomolecular Monolayers by Using N-Hydroxysuccinimide-Modified, Carboxyl-Terminated Dendrimers

The dendrimers modified with N-hydroxysuccinimide were synthesized using carboxyl-terminated dendrimers to enable the interaction with amine groups of lysine on protein surface and prepare protein monolayer on the preformed dendrimer monolayer. For this reaction, the salt was removed from the stabilized dendrimers purchased and the resulting carboxyl chain-end groups of dendrimers were modified with N-hydroxysuccinimide in organic solvent.

Separately, silicon wafer with evaporated gold was washed and then immersed in 10 mM aqueous cystamine dihydrochloride solution for 2 hours to form amine-terminated self-assembled monolayer. After reaction was finished, the film was washed with distilled water and coupling reaction was performed in a solution containing 100 μM dendrimer modified with N-hydroxysuccinimide prepared above to obtain dendrimer monolayer. Since the excessive N-hydroxysuccinimide which did not react with dendrimers is highly reactive with amine, groups on protein molecules, it was blocked and removed. Finally, the protein monolayer was prepared by adding 45 μM protein solution to the N-hydroxysuccinimide-modified dendrimer monolayer. In the present invention, the protein monolayer was prepared using model proteins including glucose oxidase, cytochrome C, and anti-biotin antibody. Fluorescence microscopic observation of the protein monolayer prepared above demonstrated that the high-density protein monolayer with uniformly spreaded biomolecules was formed.

### EXAMPLE 5

### Preparation of a Microarray of Biomolecules

For the preparation of biomolecular microarray, micropattern of dendrimers was first prepared by using microcontact printing method according to the method of Dr. Whitesides at Harvard University with modifications. Four by four (4 lines, 4 columns) arrays of dendrimer micropatterns with 100 μm×100 μm and 50 μm×50 μm, respectively were prepared and used for preparing a microarray of biomolecules of the invention.

After washing the evaporated gold surface, on which the self-assembled monolayer of mercaptoundecanoic acid was prepared. And then, terminal carboxyl groups of self-assembled monolayer were converted into amine-reactive ester form using EDAC (1-ethyl-3-(3-dimethylaminopropyl) carbodiimide hydrochloride) and pentafluorophenol. The resulting activated SAM was dried under argon atmosphere and used for printing. PDMS (poly (dimethylsiloxane)) stamp with the engraved pattern was coated with amine-terminated dendrimer solution (inking) and then brought into contact with the activated SAM to perform microcontact printing. After the stamp was peeled off, the printed surface was washed with a solvent, and micropattern of dendrimers was prepared. For example, in the present invention, terminal amine groups of dendrimers were modified with biotin-N-succinimide ester. The biotinylated microarray in this way was exposed to a solution of FITC-labeled avidin and subject to fluorescence microscopy wherein microarray of biomolecules was found to be formed with high resolution (<5 μm).

As clearly illustrated and demonstrated above, the present invention provides a process for preparing monolayers and microarrays of biomolecules by reacting functionalized dendrimers on a solid surface with biomolecules such as proteins, antigens, antibodies, enzymes, ligands, receptors, and the like. The present invention can be widely applied to the areas including preparation of kits and biosensors for disease diagnosis and compound analyses using the ascribed biomolecules as target substances, and more recently, integrated high-throughput analyzing system such as the development of protein chips.

It will apparent to those skilled in the art that certain changes and modifications can be made to this invention without departing from the spirit or scope of the invention as it is set forth herein.

What is claimed is:

1. A method of producing a patterned array of biomolecules, the method comprising:
providing a substrate comprising a surface;
derivatizing the surface with non-biomolecules;
selectively attaching dendrimers to areas of the surface so as to form a micropatterned layer of dendrimers with substantially no biomolecules between the surface and the dendrimer layer;
attaching biomolecules to the dendrimer layer so as to form a biomolecule layer over the dendrimer layer; and
wherein no additional dendrimer layer is formed over the biomolecule layer.

2. The method of claim 1, wherein the selective attachment comprises:
providing a stamp with an engraved micropattern coated with dendrimers; and
contacting the dendrimer-coated micropattern with the derivatized surface.

3. The method of claim 2, further comprising functionalizing the micropatterned layer of dendrimers so as to facilitate the biomolecule attachment.

4. The method of claim 3, wherein the functionalization comprises attaching biotin to the micropatterned dendrimer layer.

5. The method of claim 4, wherein the functionalization further comprises reacting avidin with the biotin attached to the micropatterned dendrimer layer.

6. The method of claim 3, wherein the biomolecules are modified as reactive to the functionalized micropatterned dendrimer layer.

7. The method of claim 6, wherein the biomolecules are modified to include a biotin moities.

8. A biosensor comprising a patterned array of biomolecules, the biosensor comprising:
a substrate having a surface;
a patterned layer of dendrimers over the surface with substantially no intervening biomolecules between the surface and the dendrimer layer; and
a layer of biomolecules formed over the patterned dendrimer layer, wherein no additional dendrimer layer is formed over the biomolecule layer.

9. The biosensor of claim 8, wherein the substrate is made of a material selected from the group consisting of a metal, glass and semiconductor.

10. The biosensor of claim 8, wherein the biomolecules are one or more molecules selected from the group consisting of proteins, glycoproteins, antigens, antibodies, enzymes, receptors, and ligands.

11. The biosensor of claim 8, wherein the layer of dendrimers is selectively formed over the surface.

12. The biosensor of claim 8, wherein the layer of biomolecules is selectively formed over the dendrimer layer.
